(19) United States
(12) Patent Application Publication
(10) Pub. No.: US 2016/0270671 A1
(43) Pub. Date: Sep. 22, 2016

(54) SENSOR REGIME SELECTION AND IMPLEMENTATION

(71) Applicant: Empire Technology Development LLC, Wilmington, DE (US)

(72) Inventors: Raghuram MADABUSHI, Seattle, WA (US); David ROSENBERG, San Francisco, CA (US); Michael John NICHOLLS, Freemans Reach (AU); Mark Loren GRIFFIN, Fairfax, CA (US)

(21) Appl. No.: 14/660,437

(22) Filed: Mar. 17, 2015

(51) Int. Cl. A61B 5/0205; G01C 25/00; A61B 7/04; A61B 5/11; A61B 5/145; G01C 23/00; A61B 5/00

(52) U.S. Cl. CPC A61B 5/02055; G01C 23/00; G01C 25/00; A61B 5/4875; A61B 5/112; A61B 5/4866; A61B 5/681; A61B 5/14532; A61B 5/14542; A61B 5/0077; A61B 5/1112; A61B 5/1121; A61B 7/04; A61B 5/72; A61B 5/7475; A61B 5/0002; A61B 5/01

(57) ABSTRACT

In some examples, a device may include an orientation sensor, a device sensor, a sensor regime storage unit, an analysis module, and a device output module. The orientation sensor may generate orientation data indicative of a physical state of the device. The device sensor may generate device data. The sensor regime storage unit may store sensor regimes that process the generated device data while in the physical state. The analysis module may be coupled to the orientation sensor and the sensor regime storage unit, and may determine the physical state based on the generated orientation data and select a particular sensor regime based on the determined physical state. The device output module may be coupled to the analysis module and the device sensor, and may receive the particular sensor regime and process the device data using the particular sensor regime. The device may be implemented as a wearable sensor device.

### BACKGROUND

Unless otherwise indicated herein, the materials described herein are not prior art to the claims in the present application and are not admitted to be prior art by inclusion in this section.

Some wearable devices may be limited to use at a particular body location and/or in a particular orientation. By limiting the body location and the orientation, proper contact between a body of the user and a sensor in the wearable device and consistent data collection may be possible. However, location and orientation inflexibility may limit usability and versatility of the wearable devices. Use of the wearable device at another body location and/or another orientation may result in poorly processed data and/or inaccurately generated data.

### SUMMARY

Techniques described herein generally relate to sensor regime selection and implementation.

In some examples, a device may include an orientation sensor, a device sensor, a sensor regime storage unit, an analysis module, and a device output module. The orientation sensor may be configured to generate orientation data that may be indicative of a physical state of the device. The device sensor may be configured to generate device data. The sensor regime storage unit may be configured to store multiple sensor regimes that may be configured to process the generated device data while the device is in the physical state. The analysis module may be coupled to the orientation sensor and the sensor regime storage unit. The analysis module may be configured to determine the physical state of the device based on the generated orientation data and to select a particular sensor regime from the sensor regimes based on the determined physical state. The device output module may be coupled to the analysis module and the device sensor. The device output module may be configured to receive the particular sensor regime and to process the device data using the particular sensor regime.

In some examples, a method may include determining, by one or more processors, a physical state of a device based on orientation data that are generated by one or more orientation sensors. The method may include selecting, by the one or more processors, a particular sensor regime of multiple sensor regimes based at least partially on the determined physical state of the device. The particular sensor regime may be configured to process device data that may be generated while the device is in the physical state. The method may include modifying at least one operating parameter of a device sensor in accordance with the selected particular sensor regime. The method may include generating the device data, by a device sensor modified in accordance with the particular sensor regime. The method may include processing, by the one or more processors, the device data using the selected particular sensor regime to produce output data.

In some examples, a system may include a device. The device may include an orientation sensor, a device sensor, a sensor regime storage unit, a calibration storage unit, a processor, and a non-transitory computer-readable medium. The orientation sensor may be configured to generate orientation data. The device sensor may be configured to generate device data. The sensor regime storage unit may be configured to store multiple sensor regimes that may be configured to process the device data that is generated while the device is in a physical state. The calibration storage unit may be configured to store one or more calibration data sets indicative of possible physical states of the device. The processor may be coupled to the sensor regime storage unit, the calibration storage unit, the orientation sensor, and the device sensor. The non-transitory computer-readable medium may be coupled to the processor. The non-transitory computer-readable medium may include computer-readable instructions stored thereon, which in response to execution by the processor, cause the processor to perform or cause the processor to control performance of operations. The operations may include comparing a subset of the generated orientation data to one or more of the stored calibration data sets. The operations may include determining the physical state of the device based on the comparison. The operations may include selecting a particular sensor regime of the stored sensor regimes based at least partially on the determined physical state. The operations may include modifying at least one operating parameter of a device sensor according to the selected particular sensor regime. The operations may include processing the generated device data using the selected particular sensor regime to produce the output data.

In some examples, a wearable sensor device may include a first sensor, a second sensor, and an analysis module. The first sensor may include a sensor surface and may be configured to sense a biological condition via the sensor surface. The second sensor may be configured to sense whether the sensor surface faces towards a body of a user or faces away from the body of the user. The analysis module may be coupled to the second sensor. The analysis module may be configured to select a first sensor regime in response to the second sensor having sensed that the sensor surface faces towards the body of the user. The analysis module may be configured to select a second sensor regime in response to the second sensor having sensed that the sensor surface faces away from the body of the user. In the first sensor regime, the biological condition may be automatically and repeatedly sensed by the first sensor absent a prompt by the user to sense the biological condition. In the second sensor regime, the biological condition may be sensed by the first sensor in response to a prompt by the user, including finger contact on the sensor surface by the user.

In some examples, a method to manufacture a wearable sensor device may include generating calibration data sets. The calibration data sets may be indicative of possible physical states of the wearable sensor device. The method may include storing the calibration data sets in a calibration storage unit. The method may include generating sensor regimes. The sensor regimes may be configured to process device data that may be generated while the wearable sensor device is in or subject to a particular physical state. The method may include storing the sensor regimes in a sensor regime storage unit. The method may include embedding a first sensor in a circuit board. The first sensor may be configured to sense a biological condition in two or more physical states. The method may include coupling the first sensor, a second sensor, the calibration storage unit, and the sensor regime storage unit to an analysis module. The method may include encasing the circuit board in a housing. The method may include attaching the housing to a flexible strap. The flexible strap may enable the wearable sensor device to be used in the two or more physical states.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings. In the drawings:

FIG. 1A illustrates an example system in which a device may be implemented;

FIG. 1B illustrates another example system in which the device may be implemented;

FIG. 2 illustrates an example embodiment of the device of FIGS. 1A and 1B;

FIGS. 3A and 3B illustrate an example wearable sensor device that may be implemented in the systems of FIGS. 1A and 1B;

FIG. 4 illustrates an example plot of an example first sensor regime, an example second sensor regime, and an example third sensor regime that may be implemented in the device of FIGS. 1A-2 or the wearable sensor device of FIGS. 3A and 3B;

FIGS. 5A and 5B illustrate a flow diagram of an example method to produce output data;

FIG. 6 illustrates an example method to manufacture a wearable sensor device; and

FIG. 7 is a block diagram illustrating an example computing device that is arranged to select and implement sensor regimes,

all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and computer program products related to sensor regime selection and implementation.

Briefly stated, in some examples, a device may be configured to produce output data in two or more physical states and/or while subject to two or more environmental conditions. The device may select a sensor regime that may be configured to process device data while the device is in the two or more physical states and/or while subject to the two or more environmental conditions. The device may include an orientation sensor and/or an environmental sensor that may generate orientation data and environmental data, respectively. A particular physical state and/or a particular environmental condition may be determined based on the generated orientation data and/or the generated environmental data. The sensor regime may be selected based on the determined physical state and/or the determined environmental condition. When selected, the sensor regime may be used to modify operating parameters of a device sensor of the device and/or to determine particular processing characteristics of device data generated by the device sensor. Using the sensor regime, the device may produce output data.

In some embodiments, the device may include a wearable sensor device. Additionally, in these and other embodiments, the device may be configured to produce output data that may represent a biological condition of a user (e.g., a wearer). Some examples of the biological condition may include a heart rate, a hydration level, a perspiration level, a body temperature, a respiratory rate, an activity level, a stress level, or another biological condition. In some embodiments, the device may include another sensor device. The device may be configured to measure one or more conditions of an apparatus, an animal, a piece of equipment, an environment, a vehicle, or others or combinations thereof.

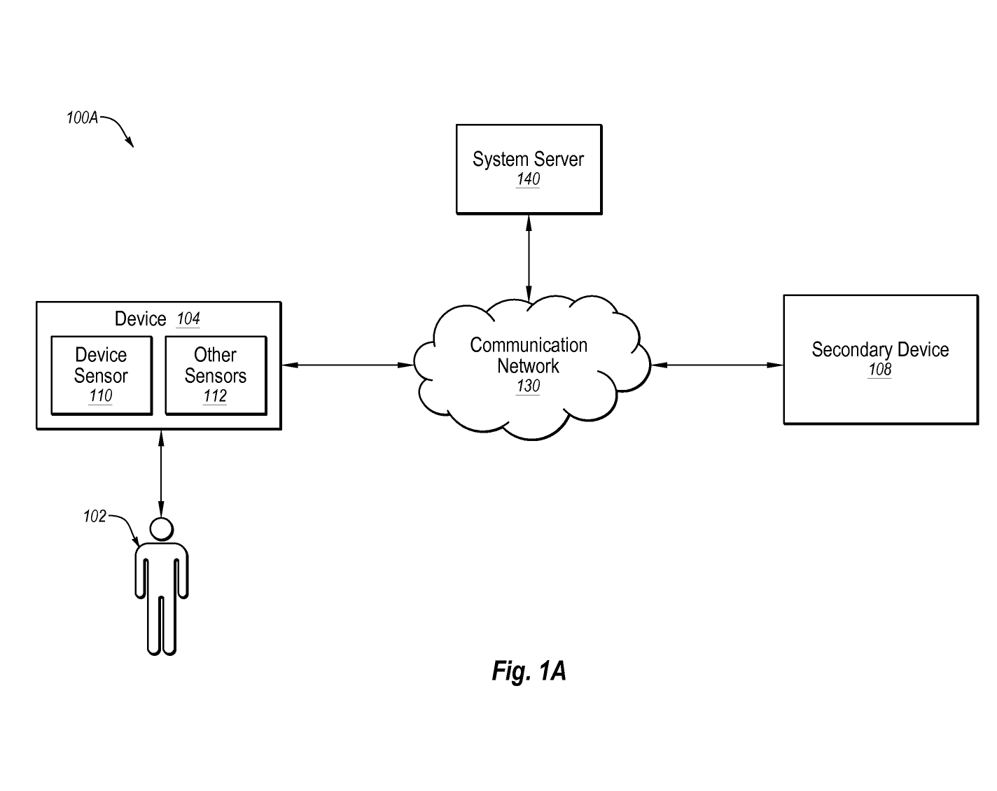

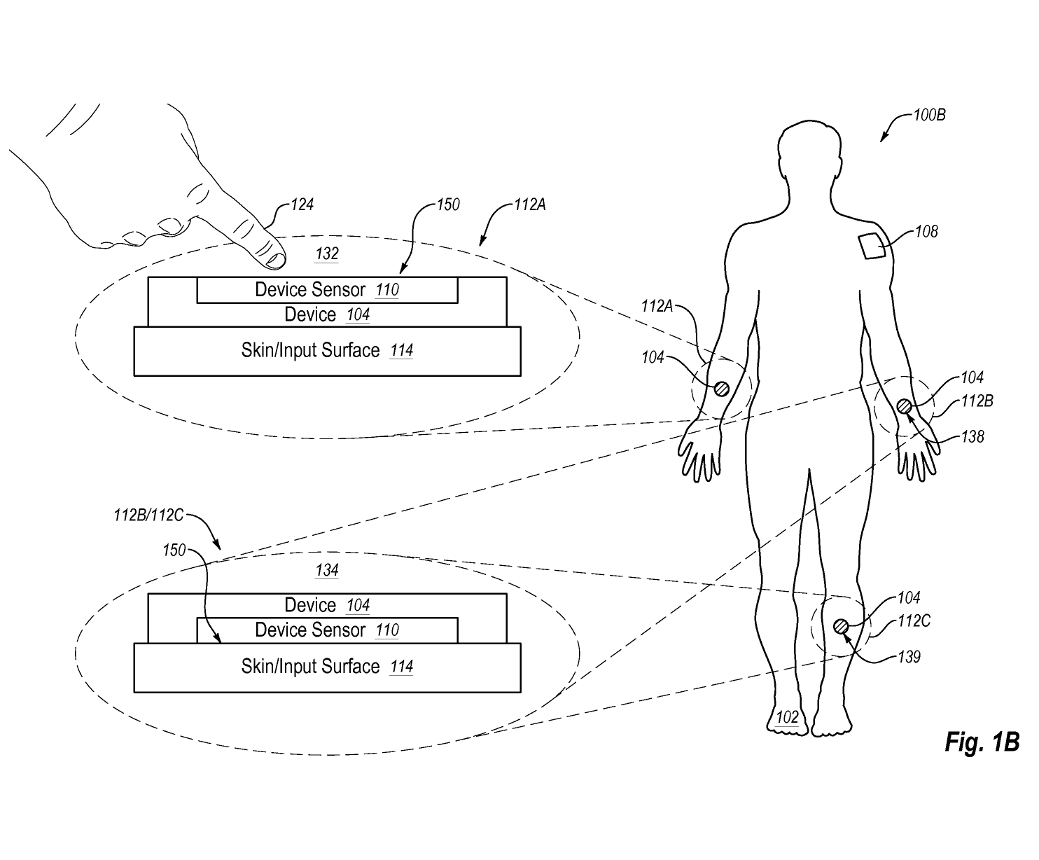

FIG. 1A illustrates an example system 100A in which a device 104 may be implemented, arranged in accordance with at least some embodiments described herein. The example of the device 104 may be configured to generate output data. The output data may be based on device data generated by a device sensor 110 included in the device 104. Some examples of the device sensor 110 may include one or more of a hydration sensor, a thermometer, an oximeter, a heart rate monitor, a biosensor, a pedometer, a calorimeter, a watch, a biosensor, an accelerometer, a strain gauge, a blood glucose sensor, an oxygen sensor, an optical sensor, a heart rate monitor, a moisture sensor, a positional sensor, a rotational sensor, a pressure sensor, a force sensor, a camera, a microphone, or other types of sensors or combinations thereof.

The device 104 may be configured to process the device data in two or more physical states. The physical states may include orientations of the device 104 and placements of the device 104, for instance. Additionally or alternatively, the device 104 may be configured to process the device data generated by the device sensor 110 while the device 104 is subject to two or more environmental conditions. The environmental conditions may include a device altitude, an ambient weather condition, in vivo versus in vitro implementation, an ambient temperature within a temperature range, an ambient pressure within a pressure range, an ambient humidity, or other environmental conditions or combinations thereof.

To process the device data, the device 104 may implement a sensor regime. The sensor regime may be configured to process the device data that is generated while the device 104 is in a particular physical state and/or subject to a particular environmental condition. Generally, the particular physical state may include a current physical state of the device 104. Similarly, the particular environmental condition may include a current environmental condition of the device 104.

The device 104 may include one or more other sensors 112. The other sensors 112 may be configured to generate data that may be indicative of the particular physical state and/or the particular environmental condition. The device 104 may compare a subset of the data generated by the other sensors 112 to one or more calibration data sets. Based at least partially on the comparison between the subset of the data generated by the other sensors 112 and the calibration data sets, the device 104 may determine the particular physical state of the device 104 and/or the particular environmental condition of the device 104. The device 104 may select a particular sensor regime based at least partially on the particular physical state and/or the particular environmental condition.

The device 104 may modify an operating parameter of the device sensor 110 in some embodiments. The device 104 may modify the operating parameter according to or for consistency with the particular sensor regime. The device 104 may process the generated device data using the selected particular sensor regime. Processing the generated device data using the selected particular sensor regime may produce the output data.

In some embodiments, determination of the physical state and/or the environmental condition, selection of the particular sensor regime, modification of the at least one operating parameter of the device sensor 110, processing the device data, or some combination thereof may occur with little or no action by a user 102. For example, the device 104 may be in a first physical state. The other sensors 112 may generate data indicative of the first physical state. The device 104 may select the particular sensor regime configured to process device data while the device 104 is in the first physical state and process the device data using the particular sensor regime without action by the user 102.

Additionally or alternatively, the determination of the particular physical state and/or the particular environmental condition, the selection of the particular sensor regime, the modification of the at least one operating parameter of the device sensor 110, the processing the device data, or some combination may repeatedly occur. For example, the physical state of the device 104 may change. The other sensors 112 may generate additional data indicative of a changed physical state. The device 104 may select an alternative sensor regime configured to process device data while the device 104 is in the changed physical state and process the device data using the alternative sensor regime.

In the system 100A, the output data may be communicated via a communication network 130 to a secondary device 108 and/or a system server 140. The communication network 130 may be wired or wireless or a combination of both. The communication network 130 may include a star configuration, a token ring configuration, or another suitable configuration. The communication network 130 may include a local area network (LAN), a wide area network (WAN) (e.g., the Internet), and other interconnected data paths across which multiple devices (e.g., the device 104, the system server 140, and the secondary device 108) may communicate. In some embodiments, the communication network 130 may include a peer-to-peer network. The communication network 130 may also be coupled to or include portions of a telecommunications network that may enable communication of data in a variety of different communication protocols. In some embodiments, the communication network 130 includes BLUETOOTH® communication networks and/or cellular communications networks for sending and receiving data including via short messaging service (SMS), multimedia messaging service (MMS), hypertext transfer protocol (HTTP), direct data connection, wireless application protocol (WAP), e-mail, etc.

The system server 140 may include a hardware server that includes a processor, memory, and communication capabilities. In the illustrated embodiment, the system server 140 may be coupled to the communication network 130 to send and receive data to and from the device 104 and/or the secondary device 108. For example, the system server 140 may receive the output data. The system server 140 may store, process, display, or make available the output data, some representation thereof, or some data derived therefrom.

The secondary device 108 may include may include a computing device that includes a processor, memory, and network communication capabilities. For example, the secondary device 108 may include a mobile device, a laptop computer, a desktop computer, a smart watch, a tablet computer, a mobile telephone, a smartphone, a personal digital assistant (“PDA”), a mobile e-mail device, a portable game player, a portable music player, a television with one or more processors embedded therein or coupled thereto, or another electronic device capable of accessing the communication network 130. The secondary device 108 may receive the output data from the device 104 and/or the system server 140. The secondary device 108 may store, process, display, or make available the output data, some representation thereof, or some data derived therefrom.

Modifications, additions, or omissions may be made to the system 100A without departing from the scope of the present disclosure. For example, embodiments of the system 100A depicted in FIG. 1A include one device 104, one system server 140, and one secondary device 108. The present disclosure applies to systems 100A including one or more of the devices 104, one or more of the system servers 140, one or more of the secondary devices 108, other element(s), or any combination thereof. Moreover, the separation of the device 104, the system server 140, and the secondary device 108 in the embodiments described herein is not meant to indicate that the separation occurs in all embodiments. Additionally, it may be understood with the benefit of this disclosure that one or more of the device 104, the system server 140, the secondary device 108, or some combination thereof may be integrated together in a single component or separated into multiple components.

FIG. 1B illustrates another system 100B in which an embodiment of the device 104 of FIG. 1A may be implemented, arranged in accordance with at least some embodiments described herein. The device 104 is depicted in a first physical state 112A, a second physical state 112B, and a third physical state 112C (generally, physical state 112 or physical states 112). It may be understood with the benefit of this disclosure, that the physical states 112 may not occur simultaneously. The physical states 112 may occur during different periods of use of the device 104.

The first physical state 112A may include a first orientation 132. For example, the first physical state 112A may include the first orientation 132 in which a sensor surface 150 of the device sensor 110 is oriented away from a skin/input surface 114 of the user 102. The device 104 may be configured to determine the first physical state 112A and may select a first sensor regime that may be configured to process the device data while the device 104 is in the first physical state 112A.

For example, while the device 104 is in the first physical state 112A, the device data may be gathered from occasional contact between an appendage 124 of the user 102 and the sensor surface 150. The device 104 may modify an operating parameter of the device sensor 110 to gather data from the occasional contact between the appendage 124 and the sensor surface 150. The device data generated by the device sensor 110 while the device 104 is in the first physical state 112A may be processed using the selected sensor regime. The occasional contact (or regular contact in some situations) may involve, for instance, the appendage 124 (or other body part) touching the sensor surface 150 so as to enable the device sensor 110 to determine a temperature, hydration level, pulse rate, etc. of the user 102.

The second physical state 112B may include a second orientation 134 and/or a first placement 138. The second physical state 112B may include the second orientation 134 in which the sensor surface 150 is oriented towards the skin/input surface 114 of the user 102. Additionally or alternatively, the second physical state 112B may include the first placement 138 in which the device 104 is placed on an arm of the user 102. The device 104 may determine the second physical state 112B. For example, the device 104 may determine that the device 104 is oriented according to the second orientation 134 and/or is placed on the arm of the user 102. The device 104 may select a second sensor regime that may be configured to process device data while the device 104 is in the second physical state 112B.

For example, while the device 104 is in the second physical state 112B, the device data may be gathered from substantially constant and/or continuous contact (or otherwise close proximity) between the skin/input surface 114 of the user 102 and the sensor surface 150. The device 104 may modify an operating parameter of the device sensor 110 to gather data at some interval based on the contact (or otherwise close proximity) between the skin/input surface 114 and the sensor surface 150. The device data generated by the device sensor 110 while the device 104 is in the second physical state 112B may be processed using the second sensor regime.

The third physical state 112C may include the second orientation 134 and a second placement 139. The second placement 139 may include a placement on a leg of the user 102. The device 104 may determine the third physical state 112C. For example, the device 104 may determine that the device 104 is oriented according to the second orientation 134 and/or is placed on the leg of the user 102. The device 104 may select a third sensor regime that may be configured to process device data while the device 104 is in the third physical state 112C.

Similar to second physical state 112B, while the device 104 is in the third physical state 112B, the device data may be gathered from substantially constant and/or continuous contact (or otherwise close proximity) between the skin/input surface 114 and the sensor surface 150. The device 104 may modify an operating parameter of the device sensor 110 to gather data at some interval based on the contact (or otherwise close proximity) between the skin/input surface 114 and the sensor surface 150. In addition, there may be a difference between device data generated by the device sensor 110 when placed on the second placement 139 as opposed to when the device 104 is placed on the first placement 138. The device sensor 110 may be modified to account for such differences based on the third sensor regime.

Additionally, the device 104 may be configured to determine a change in physical state. For example, the device 104 may be configured to determine that the device 104 has changed from the first physical state 112A to the second physical state 112B or from the second physical state 112B to the third physical state 112C. Determination of the change in physical state may be performed without action by the user 102 following an action that physically changes the state of the device 104. For example, the user 102 may move the device 104 from the first placement 138 to the second placement 139. The device 104 may determine that the device 104 is changed from the second physical state 112B to the third physical state 112C. Based on the change of the physical state, the device 104 may select an alternative sensor regime and process device data generated by the device sensor 110 using the alternative sensor regime. As an example implementation, the first physical state 112A may enable a situation where the user 102 affirmatively or consciously places the appendage 124 in contact with the exposed sensor surface 150, in order for the device sensor 110 to take a sensor reading from the appendage 124. The device 104 may be operating in a sensor regime associated with the first physical state 112A such that the sensor regime configures the device 104 to prompt the user 102 to contact the sensor surface 150 or to otherwise await the user 102 to contact the sensor surface 150, before a reading by the device sensor 110 is taken. For an example implementation for the second physical state 112B or the third physical state 112C, the sensor surface 150 is in contact with the skin/input surface 114, such that no affirmative or conscious user action need be used in order for the device sensor 110 to take a reading—the sensor regime for the second physical state 112B or the third physical state 112C may configure the device 104 to take a reading by the device sensor 110 automatically (and repeatedly, if appropriate) without a prompt or an affirmative/conscious user action.

As discussed with reference to FIG. 1A, the device 104 may generate the output data. The output data may be communicated from the device 104 to the secondary device 108. For example, the device 104 may include a wearable hydration sensor device. The output data may include data representative of a hydration level of the user 102. The data representative of the hydration level may be communicated to the secondary device 108 via a communication network such as the communication network 130 of FIG. 1A. The secondary device 108 may then display the data representative of the hydration level of the user 102 or some data derived therefrom, for instance.

In FIG. 1B, three physical states 112 are depicted. In some embodiments, the device 104 may be configured to process device data in fewer than three or more than three physical states 112. Accordingly, more than three or fewer than three sensor regimes may exist that may be configured to process data while the device 104 is in each of the physical states 112.

Additionally, the examples discussed with reference to FIG. 1B may be based on the physical states 112. In some embodiments, the device 104 may determine which of one or more environmental conditions to which the device 104 is subject. The sensor regime may be selected based on the environmental condition. In some embodiments, the sensor regime may be selected based on a particular environmental condition and a particular physical state of the device 104.

Additionally or alternatively, the sensor regime may be selected based on one or more characteristics of the user 104. The characteristics of the user 104 may be determined by the device 104, input by an administrative entity, or may be set by the user 102. The characteristics of the user 102 may include a demographic attribute of the user 102 such as an address, an age, a gender, a disability, and the like. Additionally or alternatively, the characteristic of the user 102 may include a physical characteristic of the user 102 such as a height, a weight, a fitness level, and the like.

The embodiments discussed with reference to FIG. 1B include the device 104 that includes the sensor surface 150 that measures input through contact with the skin/input surface 114 or the appendage 124. In some embodiments, the device sensor may include a pedometer, a calorimeter, a watch, a biosensor, an accelerometer, a strain gauge, a blood glucose sensor, an oxygen sensor, an optical sensor, or a heart rate monitor, for instance, that may measure input in the same or a different manner.

FIG. 1B depicts the device 104 in the three physical states 112 that are each associated with the user 102. Alternatively or additionally, the device 104 may be configured to operate with a piece of equipment, an environment, a vehicle, or an animal, for instance.

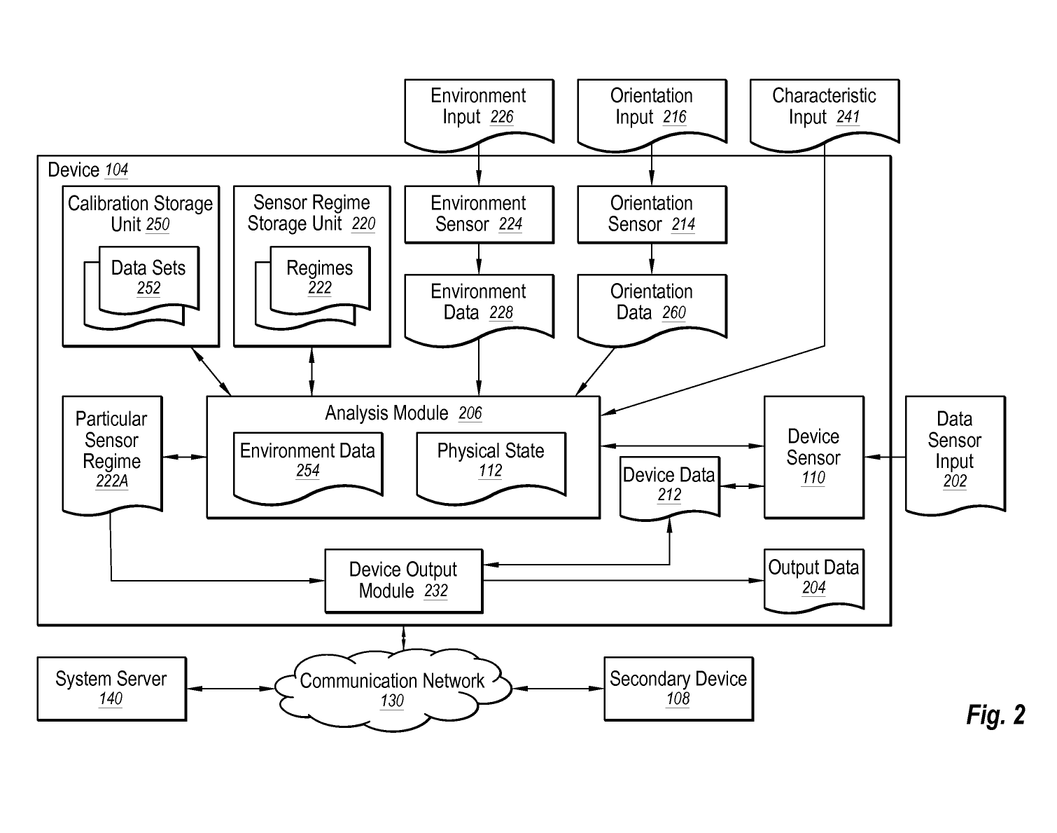

FIG. 2 illustrates an example embodiment of the device 104 of FIGS. 1A and 1B, arranged in accordance with at least some embodiments described herein. As in FIG. 1A, the device 104 may be coupled to the system server 140 and/or the secondary device 108 via the communication network 130. In general, the device 104 may be configured to generate output data 204 from device data 212. The output data 204 may be communicated to the system server 140 and/or the secondary device 108, for instance.

The device 104 may include a device output module 232 and an analysis module 206. The analysis module 206 may be coupled to the device output module 232. The analysis module 206 may be configured to select a particular sensor regime 222A and the device output module 232 may be configured to generate the output data 204 based on the particular sensor regime 222A. Although the device output module 232 and the analysis module 206 are depicted separately in FIG. 2, in some embodiments, the device output module 232 and the analysis module 206 may be included in a single module.

The device output module 232 and/or the analysis module 206 may be implemented by use of software (or other computer-executable instructions stored on a tangible non-transitory computer-readable medium) including one or more routines configured to perform one or more operations. The device output module 232 and/or the analysis module 206 may include a set of instructions executable by one or more processors to provide the functionality or operations/features, or some portion thereof, described herein. In some instances, the device output module 232 and/or the analysis module 206 may be stored in or at least temporarily loaded into memory and may be accessible and executable by the one or more processors. One or more of the device output module 232 and/or the analysis module 206 may be adapted for cooperation and communication with the one or more processors and components of the device 104 via a bus.

The device 104 may include a calibration storage unit 250. The calibration storage unit 250 may include a database or another suitable storage unit, for instance. The calibration storage unit 250 may be coupled to the analysis module 206. The calibration storage unit 250 may be configured to store one or more calibration data sets (in FIG. 2, “data sets”) 252. The calibration data sets 252 may be indicative of possible physical states (e.g., the physical states 112 of FIG. 1B) and/or possible environmental conditions of the device 104 and/or may contain or represent other information.

The device 104 may include a sensor regime storage unit 220. The sensor regime storage unit 220 may include a database or another suitable storage unit, for instance. The sensor regime storage unit 220 may be coupled to the analysis module 206. The sensor regime storage unit 220 may be configured to store one or more sensor regimes (in FIG. 2, “regimes”) 222. The sensor regimes 222 may be configured to process the device data 212 that may be generated while the device 104 is in a physical state and/or subject to an environmental condition 254. For example, the sensor regimes 222 may include one or more of: a calibration for a device sensor 110, a noise mitigation algorithm for the device data 212, a device data sample type, a device sensor measurement period, a device sensor sensitivity, a data transfer period, a sampling duration, an arithmetic function in which the device data 212 is processed, and/or other information.

The device 104 may include an environmental sensor 224 and/or an orientation sensor 214. The environmental sensor 224 and/or the orientation sensor 214 may be coupled to the analysis module 206. The environmental sensor 224 may be configured to receive, monitor, or measure environmental input 226 and generate environmental data 228 therefrom. The environmental data 228 may be communicated to the analysis module 206. Some examples of the environmental sensor 226 may include one or more or a combination of a thermometer, an altimeter, a barometer, a hydration sensor, a humidity sensor, a clock, and others.

The orientation sensor 214 may be configured to receive, monitor, or measure orientation input 216 and generate orientation data 260 therefrom. The orientation data 260 may be communicated to the analysis module 206. Some examples of the orientation sensor 214 may include one or more or a combination of a compass, an accelerometer, an optical sensor, a proximity sensor, a thermometer, a pressure sensor, a force sensor, a camera, a microphone, a microphone, a gyroscope, and others.

In some embodiments, the orientation sensor 214 may be configured to sense an orientation of a particular component of the device 104. For example, with combined reference to FIGS. 1B and 2, the orientation sensor 214 may be configured to generate the orientation data 260 that is representative of whether the sensor surface 150 faces towards the skin/input surface 114 as in the second orientation 134 or away from the skin/input surface 114 as in the first orientation 132.

Referring back to FIG. 2, the analysis module 206 may be configured to compare a subset of the orientation data 260 and/or a subset of the environmental data 228 to one or more of the calibration data sets 252. Based on the comparison, the analysis module 206 may determine the physical state 112 and/or the environmental condition 254 of the device 104. The analysis module 206 may select the particular sensor regime 222A of the sensor regimes 222 based at least partially on the particular physical state and/or the particular environmental condition 254.

As mentioned above, in addition, the particular sensor regime 222A may be selected based on a characteristic of a user. In these and other embodiments, characteristic input 241 may be further input to the analysis module 206. Additionally or alternatively, the characteristic input 241 may be represented in the environmental data 228 and/or the orientation data 260.

In some embodiments, the calibration data sets 252 and/or the sensor regimes 222 may be generated a priori or preset. Additionally, the calibration data sets 252 and/or the sensor regimes 222 may be periodically updated. After the calibration data sets 252 and/or the sensor regimes 222 are generated, the calibration data sets 252 and/or the sensor regimes 222 may be stored in the calibration storage unit 250 and the sensor regime storage unit 220, respectively.

For example, a manufacturer of the device 104 may determine the possible physical states and/or the possible environmental conditions of the device 104. The manufacturer may place the device 104 in one of the possible physical states and/or expose the device 104 to one of the possible environmental conditions. During placement of the device 104 in the possible physical state, the orientation data 260 generated by the orientation sensor 214 may be collected and stored as one of the calibration data sets 252. Similarly, during exposure of the device 104 to the possible environmental conditions, the environmental data 228 generated by the environmental sensor 224 may be collected and stored as one of the calibration data sets 252. The calibration data sets 252 may be similarly generated for one or more other physical states of the possible physical states and/or one or more other environmental conditions of the possible environmental conditions.

Additionally or alternatively, during placement of the device 104 in the one of the possible physical conditions, one or more of the sensor regimes 222 may be generated. The manufacturer of the device 104 may process the device data 212 while the device 104 is in one of the possible physical conditions to develop the particular sensor regime 222A for the one of the possible physical conditions. For example, the manufacturer may develop one or more of: the calibration for a device sensor 110, the noise mitigation algorithm for the device data 212, the device data sample type, the device sensor measurement period, the device sensor sensitivity, the data transfer period, the sampling duration, and the arithmetic function in which the device data 212 is processed and/or other parameters or combinations thereof. The sensor regimes 222 may be similarly generated for one or more other physical states of the possible physical states and/or one or more other environmental conditions of the possible environmental conditions.

The analysis module 206 may be configured to modify at least one operational parameter of a device sensor 110 according to the particular sensor regime 222A. For example, the calibration for the device sensor 110, the noise mitigation algorithm for the device data 212, the device data sample type, the device sensor measurement period, the device sensor sensitivity, the data transfer period, the sampling duration, and the arithmetic function in which the device data 212 is processed and/or other parameters or combinations thereof may be modified.

The analysis module 206 may communicate the particular sensor regime 222A to the device output module 232. The device output module 232 may receive the device data 212 from the device sensor 110. The device data 212 may be generated based on data sensor input 202 that may be measured or otherwise obtained by the device sensor 110. The device output module 232 may process the device data 212 using the particular sensor regime 222A to produce the output data 204.

The environmental sensor 224 may generate additional environmental data (similar to the environmental data 228). The orientation sensor 214 may generate additional orientation data (similar to the orientation data 260). The additional environmental data and/or the additional orientation data may be communicated to the analysis module 206. The additional environmental data and/or the additional orientation data may be compared to the calibration data sets 252. From the comparison between additional environmental data and/or the additional orientation data and the calibration data sets 252, the analysis module 206 may determine whether the physical state and/or the environmental condition is changed.

In response to a determination that the physical state and/or the environmental condition are unchanged, the device output module 232 may continue to process the device data 212 using the particular sensor regime 222A. In response to a determination that the physical state and/or the environmental condition is changed, the analysis module 206 may select an alternative sensor regime (similar to the particular sensor regime 222A) of the sensor regimes 222. The alternative sensor regime may be communicated to the device output module 232. The device output module 232 may process the device data 212 using the alternative sensor regime.

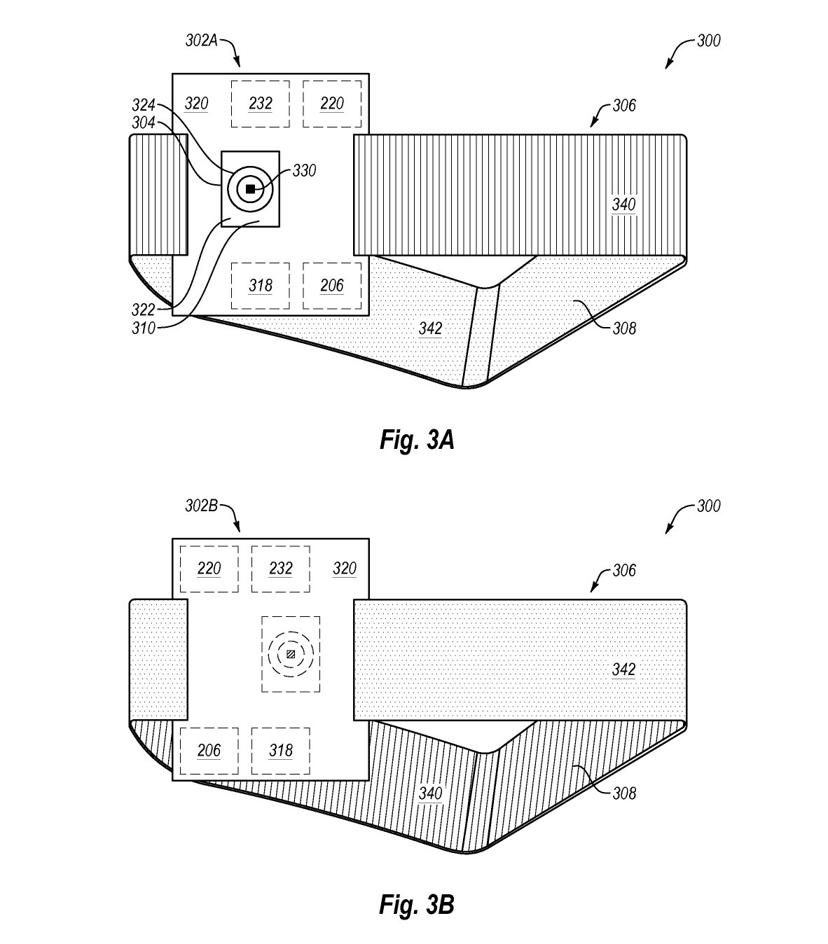

FIGS. 3A and 3B illustrate an example wearable sensor device 300 that may be implemented in the systems 100A and 100B of FIGS. 1A and 1B, arranged in accordance with at least some embodiments described herein. The wearable sensor device 300 of FIGS. 3A and 3B may include an example of the device 104 discussed with reference to FIGS. 1A-2. Generally, a first sensor 304 of the wearable sensor device 300 may be configured to sense a biological condition via a sensor surface 310. The wearable sensor device 300 may be used in at least a first physical state 302A, which is depicted in FIG. 3A, and in a second physical state 302B, which is depicted in FIG. 3B. In the first physical state 302A, the sensor surface 310 of the first sensor 304 may be positioned such that the sensor surface 310 faces away from a body of a user. The body or a portion thereof of the user (such as an arm, leg, etc.) may be positioned in an opening 306 defined by a flexible strap 308 of the wearable sensor device 300, for instance. In the second physical state 302B, the sensor surface 310 of the first sensor 304 may face the body of the user. In FIG. 3B, the first sensor 304 is depicted with dashed lines, which indicates that the first sensor 304 faces the opening 306.

In the embodiment depicted in FIGS. 3A and 3B, the wearable sensor device 300 may include one sensor surface 310. In some embodiments, the wearable sensor device 300 may include multiple sensor surfaces that may be substantially similar to the sensor surface 310. In these and other embodiments, the wearable sensor device 300 may be configured to sense a biological condition using one or more of the multiple sensor surfaces.

The first sensor 304 may include one or more rings 324 and/or a lead 330. The rings 324 and the lead 330 may be positioned on the sensor surface 310. The rings 324 and the lead 330 may be configured to measure hydration levels using the sensor surface 310 or another biological condition. The rings 324 and the lead 330 may be embedded in a circuit board 322 or a flexible circuit material.

In FIGS. 3A and 3B, the rings 324 may include two substantially concentric rings. In some embodiments, there may be more than two rings and/or the rings 324 may include differing positions relative to one another. Additionally, in FIGS. 3A and 3B, the lead 330 may be positioned within the rings 324. In some embodiments, the lead 330 may be positioned in another location on the sensor surface 310. Additionally or alternatively, some embodiments may include multiple leads 330.

The circuit board 322 may be encased, at least partially, in a housing 320. Additionally, the device output module 232, the analysis module 206, the sensor regime storage unit 220, and a second sensor 318 may be positioned, at least partially, in the housing 320 or otherwise coupled to the housing. The device output module 232, the analysis module 206, the sensor regime storage unit 220, and the second sensor 318 are depicted with a dashed border to indicate examples of the position within the housing 320. The device output module 232, the analysis module 206, the sensor regime storage unit 220, and the second sensor 318 may be communicatively coupled to each other.

The device output module 232 may be configured to generate the output data that is based on the biological condition sensed by the first sensor 304. The device output module 232 may process the generated output data based on whether the wearable sensor device 300 is in the first physical state 302A or in the second physical state 302B.

The flexible strap 308 may be attached to the housing 320. The flexible strap 308 may enable the wearable sensor device 300 to be used in the first physical state 302A and the second physical state 302B. For instance, the flexible strap 308 may include a front surface 340 and a back surface 342. When the wearable sensor device 300 is in the first physical state 302A as in FIG. 3A, the front surface 340 of the flexible strap 308 and the sensor surface 310 may face away from the body of the user. Additionally, the back surface 342 may face towards the body of the user. Conversely, when the wearable sensor device 300 is in the second physical state 302B as in FIG. 3B, the front surface 340 of the flexible strap 308 and the sensor surface 310 may face towards the body of the user. Additionally, the back surface 342 may face away the body of the user.

The flexible strap 308 may include multiple lengths, which may be stretchable and/or adjustable. For example, in some embodiments, the flexible strap 308 may adjust to about four inches such that the flexible strap 308 may be used on a wrist of the user. Alternatively or additionally, the flexible strap 308 may be adjusted to about twenty-nine inches such that the flexible strap 308 may be used around a chest of the user.

In FIGS. 3A and 3B, the housing 320 may be attached to the flexible strap 308. In some embodiments, the housing 320 may be attached to a band, a clip, another suitable attachment, or some combination thereof. The band, the clip, the other suitable attachment may enable the wearable sensor device 300 to be used in the first physical state 302 and the second physical state 302B.

The second sensor 318 may be configured to sense whether the sensor surface 310 faces towards the body of the user as in the second physical state 302B of FIG. 3B or faces away from the body of the user as in the first physical state 302A of FIG. 3A. In some embodiments, the second sensor 318 may be similar to the environmental sensor 224 and/or the orientation sensor 214.

The sensor regime storage unit 220 may include a first sensor regime and a second sensor regime. The first sensor regime may be configured to process data generated by the first sensor 304 while the wearable sensor device 300 is in the first physical state 302A. For example, in the first sensor regime, the biological condition may be sensed by the first sensor 304 in response to an affirmative or conscious prompt by the user, including a finger contact on the sensor surface 310 by the user, for example. The second sensor regime may be configured to process data generated by the first sensor 304 while the wearable sensor device 300 is in the second physical state 302B. For example, in the second sensor regime, the biological condition may be automatically and repeatedly sensed by the first sensor 304 absent an affirmative or conscious prompt by the user to sense the biological condition.

The analysis module 206 may be coupled to the second sensor 318. The analysis module 206 may be configured to select a corresponding one of the first and second sensor regimes. For example, the analysis module 206 may select the first sensor regime in response to the second sensor 318 having sensed that the sensor surface 310 faces towards the body of the user or may select the second sensor regime in response to the second sensor 318 having sensed that the sensor surface 310 faces away from the body of the user.

Although not explicitly shown in FIGS. 3A and 3B (for the purposes of brevity and clarity), the wearable sensor device 300 may include a calibration storage unit 250 and/or other components. Additionally or alternatively, the wearable sensor device 300 may be configured to communicate with a system server such as the system server 140 of FIGS. 1A and 2 and/or a secondary device such as the secondary device 108 of FIGS. 1A-2.

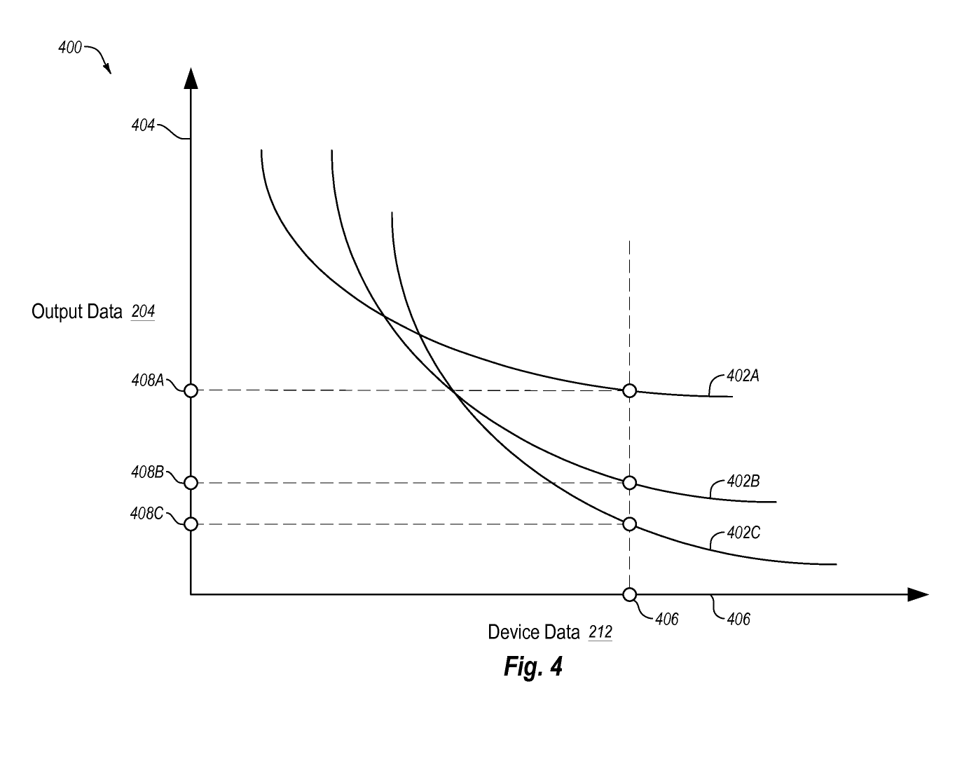

FIG. 4 illustrates an example plot 400 of an example first sensor regime 402A, an example second sensor regime 402B, and an example third sensor regime 402C that may be implemented in the device 104 of FIGS. 1A-2 or the wearable sensor device 300 of FIGS. 3A and 3B, arranged in accordance with at least some embodiments described herein. In the plot 400, a y-axis 404 corresponds to the output data 204 and an x-axis 406 corresponds to the device data 212. As described above, the device data 212 may be generated by the device sensor 110 of FIGS. 1A-2 or the first sensor 304 of FIGS. 3A and 3B, for example. The example plot 400 is purely for illustrative purposes to help describe the operation of the various embodiments of the device 104 or the wearable sensor device 300, and is not necessarily intended to precisely provide a plot of actual data/regimes. Various other plots, curvatures, behaviors, data, regime contours, etc. are possible amongst the embodiments.

The first sensor regime 402A, the second sensor regime 402B, and the third sensor regime 402C may be selected based on environmental data and/or orientation data (e.g., the environmental data 224 and/or the orientation data 260 of FIG. 2). Depending on which of the first sensor regime 402A, the second sensor regime 402B, or the third sensor regime 402C is selected, the output data 204 may change.

For example, a particular device data 406 may be generated. If the first sensor regime 402A is selected, then a first output data 408A may be output. If the second sensor regime 402B is selected, then a second output data 408B may be output. If the third sensor regime 402C is selected, then a third output data 408C may be output.

In addition to the first, second, and third sensor regimes 402A, 402B, and 402C potentially affecting the output data 204, the first, second, and third sensor regimes 402A, 402B, and 402C may affect a noise mitigation algorithm for the device data 212, a device data sample type, a device sensor measurement period, a device sensor sensitivity, a data transfer period, a sampling duration, other factors, or some combination thereof.

In some embodiments, the noise mitigation algorithm for the device data 212 may depend on which of the first sensor regime 402A, the second sensor regime 402B, and the third sensor regime 402C is selected. For example, with combined reference to FIGS. 1B and 4, when the device 104 is in the first physical state 112A, the device data 212 may include a first type (e.g., frequency or amplitude) of noise and when the device 104 is in the second physical state 112B, the device data 212 may include a second type of noise. Accordingly, the first sensor regime 402A, the second sensor regime 402B, and the third sensor regime 402C may include a noise mitigation algorithm that is particularly suited to compensate for or filter the first type of noise or the second type of noise.

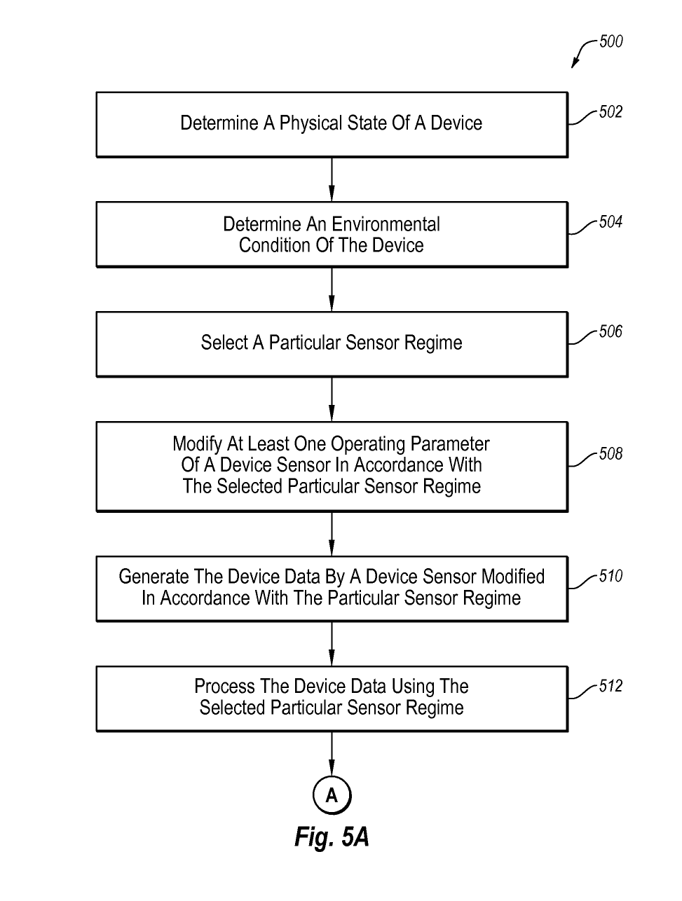

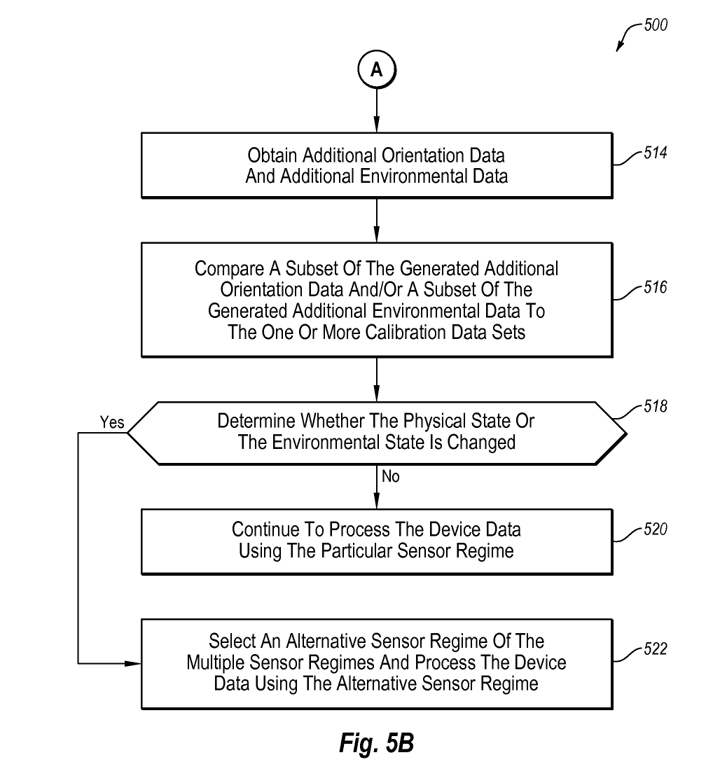

FIGS. 5A and 5B illustrate a flow diagram of an example method 500 to produce output data, arranged in accordance with at least some embodiments described herein. The method 500 may be performed, for example, in the systems 100A and 100B and/or in other systems and configurations. For example, the device 104 of FIGS. 1A-2 and/or the wearable sensor device 300 of FIGS. 3A and 3B may include an analysis module and/or an output module such as the analysis module 206 and the device output module 232 of FIG. 2 that may be configured to perform the method 500.

In some embodiments, the computing device may include or may be communicatively coupled to one or more non-transitory computer-readable media having thereon computer-readable instructions, which in response to execution by one or more processors, cause the one or more processors to perform or control performance of the method 500. The analysis module 206 and the device output module 232 in some embodiments may be implemented by such computer-readable instructions stored on one or more non-transitory computer-readable media and executable by one or more processors. Although illustrated as discrete blocks, various blocks may be divided into additional blocks, supplemented with additional blocks, combined into fewer blocks, or eliminated, depending on the particular implementation.

With reference to FIG. 5A, the method 500 may begin at block 502. At block 502 (“Determine A Physical State Of A Device”), a physical state of a device may be determined. The physical state may be determined based on orientation data that are generated by one or more orientation sensors. In some embodiments, the physical state may include an orientation of the device, a placement of the device, or both the orientation and the placement of the device. In some embodiments, the physical state of the device may be sensed by one or more orientation sensors so as to generate orientation data from the sensed orientation.

At block 504 (“Determine An Environmental Condition Of The Device”), an environmental condition of the device may be determined. In some embodiments, the environmental condition may be determined based on environmental data generated by one or more environmental sensors.

At block 506, (“Select A Particular Sensor Regime”), a particular sensor regime may be selected. For example, in some embodiments, the particular sensor regime may be selected based on the determined physical state of the device and/or on the determined environmental condition of the device. In some embodiments, selecting the particular sensor regime may include comparing a subset of the generated orientation data and/or environmental data to one or more calibration data sets. The calibration data sets may be indicative of possible physical states, possible environmental conditions of the device, a demographic attribute of a user of the device, or some combination thereof.

In some embodiments, the particular sensor regime may include one or more of a calibration for the device sensor, a noise mitigation algorithm for the device data, a device data sample type, a device sensor measurement period, a device sensor sensitivity, a data transfer period, a sampling duration, an arithmetic function in which the generated device data is processed, and/or other parameters or combinations thereof.

At block 508 (“Modify At Least One Operating Parameter Of A Device Sensor In Accordance With The Selected Particular Sensor Regime”), at least one operating parameter of a device sensor may be modified in accordance with the selected particular sensor regime. For example, the calibration for the device sensor, the noise mitigation algorithm for the device data, the device data sample type, the device sensor measurement period, the device sensor sensitivity, the data transfer period, the sampling duration, and the arithmetic function in which the generated device data is processed may be modified.

At block 510 (“Generate The Device Data By A Device Sensor Modified In Accordance With The Particular Sensor Regime”), device data may be generated by a device sensor. At block 512 (“Process The Device Data Using The Selected Particular Sensor Regime”), the device data may be processed using the selected particular sensor regime. Processing the device data may produce output data.

With reference to FIG. 5B, the method 500 may proceed to block 514. At block 514 (“Obtain Additional Orientation Data And Additional Environmental Data”), additional orientation data and/or additional environmental data may be obtained. At block 516 (“Compare A Subset Of The Generated Additional Orientation Data And/Or A Subset Of The Generated Additional Environmental Data To The One Or More Calibration Data Sets”), a subset of the generated additional orientation data and/or a subset of the generated additional environmental data may be compared to the one or more calibration data sets.

At block 518 (“Determine Whether The Physical State Or The Environmental State Is Changed”), it may be determined whether the physical state or the environmental state is changed. In response to a determination that the physical state or the environmental state is unchanged (“No” at block 518), the method 500 may proceed to block 520. In response to a determination that the physical state or the environmental condition is changed (“Yes” at block 518), the method 500 may proceed to block 522. At block 520 (“Continue To Process The Device Data Using The Particular Sensor Regime”), processing of the device data may continue using the particular sensor regime. At block 522 (“Select An Alternative Sensor Regime Of The Multiple Sensor Regimes And Process The Device Data Using The Alternative Sensor Regime”), an alternative sensor regime may be selected and the device data may be processed using the alternative sensor regime.

For this and other procedures and methods disclosed herein, the functions or operations performed in the processes and methods may be implemented in differing order. Furthermore, the outlined operations are only provided as examples, and some of the operations may be optional, combined into fewer operations, supplemented with other operations, or expanded into additional operations without detracting from the disclosed embodiments.

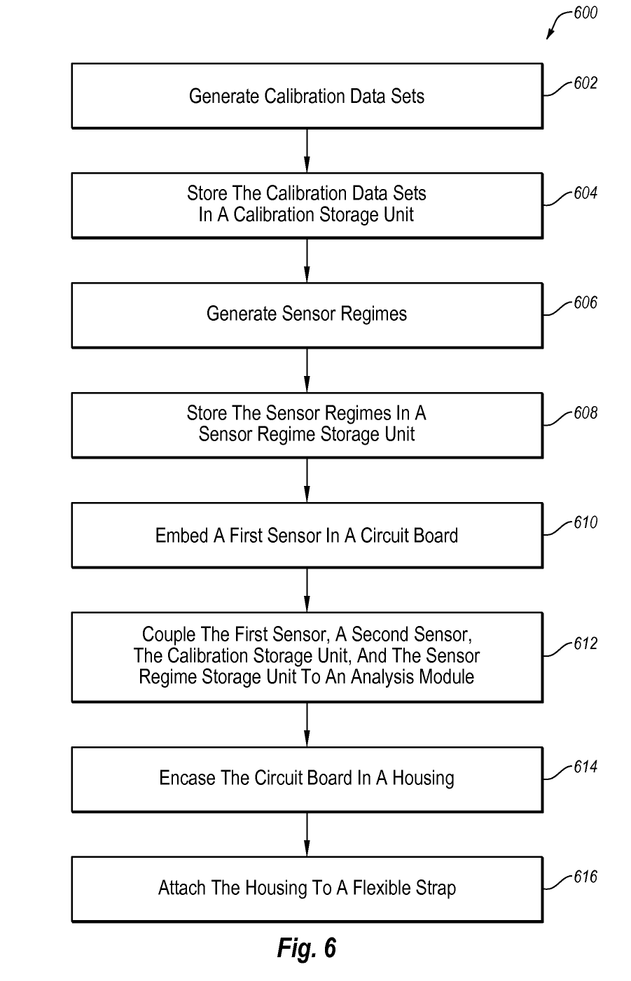

FIG. 6 illustrates an example method 600 to manufacture a wearable sensor device, arranged in accordance with at least some embodiments described herein. Although illustrated as discrete blocks, various blocks may be divided into additional blocks, combined into fewer blocks, supplemented with other blocks, or eliminated, depending on the desired implementation. The various operations can be performed in any suitable manner, and not necessarily in the specific order shown in FIG. 6. For example, it is possible to provide an embodiment wherein the manufacture and assembly of the physical components of a wearable sensor are performed first, followed by the generation of sensor regimes, calibration data sets, and/or other programming.

The method 600 may begin at block 602 (“Generate Calibration Data Sets”) in which calibration data sets may be generated. In some embodiments, the calibration data sets may be indicative of possible physical states and/or possible environmental conditions of the wearable sensor device. At block 604 (“Store The Calibration Data Sets In A Calibration Storage Unit”), the calibration data sets may be stored in a calibration storage unit. At block 606 (“Generate Sensor Regimes”), sensor regimes may be generated. In some embodiments, the sensor regimes may be configured to process device data that is generated while the wearable sensor device is in a particular physical state and/or subject to a particular environmental condition.

At block 608 (“Store The Sensor Regimes In A Sensor Regime Storage Unit”), the sensor regimes may be stored in a sensor regime storage unit. At block 610 (“Embed A First Sensor In A Circuit Board”), a first sensor may be embedded in a circuit board. In some embodiments, the first sensor may be configured to sense a biological condition in two or more physical states and/or two or more environmental conditions. The first sensor may include a sensor surface. The sensor surface may be configured to sense the biological condition via the sensor surface.

At block 612 (“Couple The First Sensor, A Second Sensor, The Calibration Storage Unit, And The Sensor Regime Storage Unit To An Analysis Module”), the first sensor, a second sensor, the calibration storage unit, and the sensor regime storage unit may be coupled to an analysis module. The second sensor may be configured to sense whether the sensor surface faces towards a body of a user or faces away from the body of the user. Additionally or alternatively, the analysis module may be configured to select a first sensor regime in response to the second sensor having sensed that the sensor surface faces towards the body of the user and to select a second sensor regime in response to the second sensor having sensed that the sensor surface faces away from the body of the user.

At block 614 (“Encase The Circuit Board In A Housing”), the circuit board may be encased in a housing. At block 616 (“Attach The Housing To A Flexible Strap”), the housing may be attached to a flexible strap. In some embodiments, the flexible strap may enable the wearable sensor device to be used in two or more physical states and/or two or more environmental conditions.

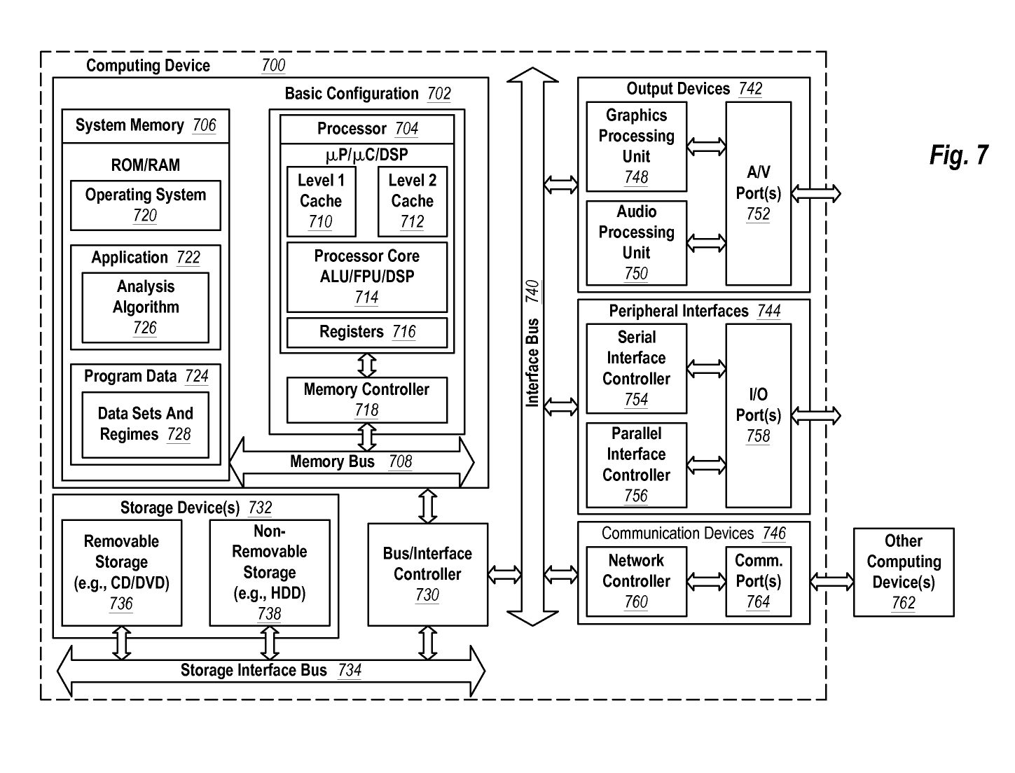

FIG. 7 is a block diagram illustrating an example computing device 700 that is arranged to select and implement sensor regimes, arranged in accordance with at least some embodiments described herein. The computing device 700 may be used in some embodiments of the device 104, the wearable sensor device 300, and/or any other device that include features and operations described herein that pertain to sensor regime selection and implementation. In a basic configuration 702, the computing device 700 typically includes one or more processors 704 and a system memory 706. A memory bus 708 may be used for communicating between the processor 704 and the system memory 706.

Depending on the desired configuration, the processor 704 may be of any type including, but not limited to, a microprocessor (μP), a microcontroller (μC), a digital signal processor (DSP), or any combination thereof. The processor 704 may include one or more levels of caching, such as a level one cache 710 and a level two cache 712, a processor core 714, and registers 716. The processor core 714 may include an arithmetic logic unit (ALU), a floating point unit (FPU), a digital signal processing core (DSP Core), or any combination thereof. An example memory controller 718 may also be used with the processor 704, or in some implementations the memory controller 718 may be an internal part of the processor 704.

Depending on the desired configuration, the system memory 706 may be of any type including, but not limited to, volatile memory (such as RAM), nonvolatile memory (such as ROM, flash memory, etc.), or any combination thereof. The system memory 706 may include an operating system 720, one or more applications 722, and program data 724. The application 722 may include an orientation and/or calibration data analysis algorithm 726 (in FIG. 7, “Analysis Algorithm 726”) that is arranged to compare orientation data and/or calibration data to calibration data sets and to select sensor regimes based thereon. The program data 724 may include values for the calibration data sets and/or the sensor regimes (in FIG. 7, “Data Sets and Regimes”) 728 as is described herein. In some embodiments, the application 722 may be arranged to operate with the program data 724 on the operating system 720 such that sensor regimes may be selected and device data may be processed using the sensor regimes as described herein. In some embodiments, the analysis algorithm 726 may be used to implement at least in part or may operate in conjunction with the analysis module 206 and/or the device output module 232.

The computing device 700 may have additional features or functionality, and additional interfaces to facilitate communications between the basic configuration 702 and any involved devices and interfaces. For example, a bus/interface controller 730 may be used to facilitate communications between the basic configuration 702 and one or more data storage devices 732 via a storage interface bus 734. The data storage devices 732 may be removable storage devices 736, non-removable storage devices 738, or a combination thereof. Examples of removable storage and non-removable storage devices include magnetic disk devices such as flexible disk drives and hard-disk drives (HDDs), optical disk drives such as compact disk (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer-readable instructions, data structures, program modules, or other data.

The system memory 706, the removable storage devices 736, and the non-removable storage devices 738 are examples of computer storage media. Computer storage media includes RAM, ROM, EEPROM, flash memory or other memory technology, CD-ROM, digital versatile disks (DVDs) or other optical storage, magnetic cassettes, magnetic tape, magnetic disk storage or other magnetic storage devices, or any other medium which may be used to store the desired information and which may be accessed by the computing device 700. Any such computer storage media may be part of the computing device 700.

The computing device 700 may also include an interface bus 740 for facilitating communication from various interface devices (e.g., output devices 742, peripheral interfaces 744, and communication devices 746) to the basic configuration 702 via the bus/interface controller 730. The output devices 742 include a graphics processing unit 748 and an audio processing unit 750, which may be configured to communicate to various external devices such as a display or speakers via one or more A/V ports 752. The peripheral interfaces 744 include a serial interface controller 754 or a parallel interface controller 756, which may be configured to communicate with external devices such as input devices (e.g., keyboard, mouse, pen, voice input device, touch input device, etc.), sensors, or other peripheral devices (e.g., printer, scanner, etc.) via one or more I/O ports 758. The communication devices 746 include a network controller 760, which may be arranged to facilitate communications with one or more other computing devices 762 over a network communication link via one or more communication ports 764.

The network communication link may be one example of a communication media. Communication media may typically be embodied by computer-readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term “computer-readable media” as used herein may include both storage media and communication media.

The computing device 700 may be implemented as a portion of a small-form factor portable (or mobile) electronic device such as a cell phone, a personal data assistant (PDA), a personal media player device, a wireless web-watch device, a personal headset device, an application-specific device, a wearable sensor device, or a hybrid device that includes any of the above functions. As noted above, at least some components of the computing device 700 may be implemented in a wearable sensor device as described herein, and/or may be communicatively coupled to a wearable sensor device. The computing device 700 may also be implemented as a personal computer including both laptop computer and non-laptop computer configurations.

The present disclosure is not to be limited in terms of the particular embodiments described herein, which are intended as illustrations of various aspects. Many modifications and variations can be made without departing from its spirit and scope. Functionally equivalent methods and apparatuses within the scope of the disclosure, in addition to those enumerated herein, will be apparent from the foregoing descriptions. Such modifications and variations are intended to fall within the scope of this disclosure. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub ranges and combinations of sub ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into sub ranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, various embodiments of the present disclosure have been described herein for purposes of illustration, and various modifications may be made without departing from the scope and spirit of the present disclosure. Accordingly, the various embodiments disclosed herein are not intended to be limiting.

What is claimed is:

1. A device, comprising:
an orientation sensor that is configured to generate orientation data indicative of a physical state of the device;
a device sensor configured to generate device data;
a sensor regime storage unit configured to store a plurality of sensor regimes that are configured to process the generated device data while the device is in the physical state;
an analysis module coupled to the orientation sensor and the sensor regime storage unit, wherein the analysis module is configured to determine the physical state of the device based on the generated orientation data and to select a particular sensor regime from the plurality of sensor regimes based on the determined physical state; and
a device output module coupled to the analysis module and the device sensor, wherein the device output module is configured to receive the particular sensor regime and to process the device data using the particular sensor regime.

2. The device of claim 1, wherein the physical state includes an orientation of the device, a placement of the device, or both the orientation and the placement of the device.

3. The device of claim 1, further comprising a calibration storage unit coupled to the analysis module, wherein:
the calibration storage unit is configured to store one or more calibration data sets,
the calibration data sets are indicative of possible physical states of the device, and
the analysis module is configured to compare a subset of the orientation data to one or more calibration data sets to determine the physical state of the device.

4. The device of claim 3, wherein the calibration data sets and the sensor regimes are preset.

5. The device of claim 1, further comprising an environmental sensor that is coupled to the analysis module, wherein the analysis module is further configured to:
determine an environmental condition of the device based on environmental data generated by the environmental sensor, and
select the particular sensor regime based at least partially on the determined environmental condition of the device.

6. The device of claim 5, wherein:
the orientation sensor includes one or more or a combination of a gyroscope, a compass, an accelerometer, an optical sensor, a proximity sensor, a thermometer, a pressure sensor, a force sensor, a camera, a microphone, and a microphone; and
the environmental sensor includes one or more or a combination of a thermometer, an altimeter, a barometer, a hydration sensor, a humidity sensor, and a clock.

7. A method, comprising:
determining, by one or more processors, a physical state of a device based on orientation data that are generated by one or more orientation sensors;
selecting, by the one or more processors, a particular sensor regime of a plurality of sensor regimes based at least partially on the determined physical state of the device, wherein the particular sensor regime is configured to process device data that is generated while the device is in the physical state;
modifying at least one operating parameter of a device sensor in accordance with the selected particular sensor regime;
generating the device data, by a device sensor modified in accordance with the particular sensor regime; and
processing, by the one or more processors, the device data using the selected particular sensor regime to produce output data.

8. The method of claim 7, wherein the determining the physical state includes determining an orientation of the device, a placement of the device, or both the orientation and the placement of the device.

9. The method of claim 7, wherein the determining the physical state includes:
sensing, by the one or more orientation sensors, an orientation of the device so as to generate the orientation data from the sensed orientation; and
comparing a subset of the generated orientation data to one or more calibration data sets that are indicative of possible physical states of the device.

10. The method of claim 9, further comprising:
generating additional orientation data from the one or more orientation sensors;
comparing a subset of the generated additional orientation data to the one or more calibration data sets;
determining whether the physical state is changed based on a comparison between a subset of the generated additional orientation data and the one or more calibration data sets;
in response to a determination that the physical state is unchanged, continuing to process the device data using the particular sensor regime; and
in response to a determination that the physical state is changed, selecting an alternative sensor regime of the plurality of sensor regimes and processing the device data using the alternative sensor regime.

11. The method of claim 9, wherein the calibration data sets are also indicative of a demographic attribute of a user of the device.

12. The method of claim 7, further comprising:
determining an environmental condition of the device based on environmental data generated by one or more environmental sensors; and
selecting the particular sensor regime based at least partially on the determined environmental condition of the device.

13. The method of claim 7, wherein selecting the particular sensor regime includes one or more of:
a calibration for a device sensor;
a noise mitigation algorithm for the device data;
a device data sample type;
a device sensor measurement period;
a device sensor sensitivity;
a data transfer period;
a sampling duration; and
an arithmetic function in which the generated device data is processed.

14. The method of claim 7, wherein the one or more orientation sensors include one or more of a gyroscope, a compass, an accelerometer, an optical sensor, a proximity sensor, a thermometer, a pressure sensor, a force sensor, a camera, a microphone, and a microphone.

15. A non-transitory computer-readable medium that includes computer-readable instructions stored thereon, which in response to execution by a processor, cause the processor to perform or cause the processor to control performance of the method of claim 7.

16. A system, comprising:
a device that includes:
an orientation sensor that is configured to generate orientation data;
a device sensor that is configured to generate device data;
a sensor regime storage unit that is configured to store a plurality of sensor regimes that are configured to process the device data that is generated while the device is in a physical state;
a calibration storage unit that is configured to store one or more calibration data sets indicative of possible physical states of the device;
a processor that is coupled to the sensor regime storage unit, the calibration storage unit, the orientation sensor, and the device sensor; and
a non-transitory computer-readable medium coupled to the processor and that includes computer-readable instructions stored thereon, which in response to execution by the processor, cause the processor to perform or cause the processor to control performance of operations that include:
compare a subset of the generated orientation data to one or more of the stored calibration data sets;
based on the comparison, determine the physical state of the device;
select a particular sensor regime of the stored plurality of sensor regimes based at least partially on the determined physical state;
modify at least one operating parameter of the device sensor according to the selected particular sensor regime; and
process the generated device data using the selected particular sensor regime to produce output data.

17. The system of claim 16, wherein:
the device further includes an environmental sensor coupled to the processor and that is configured to generate environmental data;
the one or more calibration data sets are further indicative of possible environmental conditions of the device;
the sensor regimes are further configured to process the device data that is generated while the device is also subject to an environmental condition;
the operations further comprise compare a subset of the generated environmental data to one or more calibration data sets and based on the comparison of the subset of the generated environmental data to the one or more calibration data sets, determine the physical state of the device and the environmental condition of the device; and
selection of the particular sensor regime is based at least partially on the determined environmental condition of the device.

18. The system of claim 17, wherein the operations further comprise:
obtain additional orientation data from the orientation sensor and additional environmental data from the environmental sensor;
compare a subset of the obtained additional orientation data and a subset of the additional environmental data to the calibration data sets;
determine whether the physical state or the environmental condition is changed based on the comparison of the subsets of the obtained additional orientation data and the additional environmental data to the calibration data sets;
in response to a determination that the physical state and the environmental are unchanged, continue to process the device data using the selected particular sensor regime; and
in response to a determination that the physical state or the environmental condition is changed, select an alternative sensor regime of the plurality of sensor regimes and process device data using the selected alternative sensor regime.

19. The system of claim 17, wherein:
the physical state includes an orientation of the device, a placement of the device, or both the orientation and the placement of the device; and
the environmental condition includes an ambient temperature within a temperature range, an ambient pressure within an pressure range, a device altitude, or an ambient humidity.

20. The system of claim 16, further comprising:
a system server; and
a secondary device communicatively coupled to the device and the system server via a communication network,
wherein the device is configured to communicate the output data via the communication network to the secondary device, to the system server, or to both the secondary device and the system server.

21. A wearable sensor device, comprising:
a first sensor that includes a sensor surface and that is configured to sense a biological condition via the sensor surface;
a second sensor configured to sense whether the sensor surface faces towards a body of a user or faces away from the body of the user; and
an analysis module coupled to the second sensor, wherein the analysis module is configured to select a first sensor regime in response to the second sensor having sensed that the sensor surface faces towards the body of the user and is configured to select a second sensor regime in response to the second sensor having sensed that the sensor surface faces away from the body of the user,
wherein in the first sensor regime, the biological condition is automatically and repeatedly sensed by the first sensor absent a prompt by the user to sense the biological condition, and
wherein in the second sensor regime, the biological condition is sensed by the first sensor in response to a prompt by the user, including finger contact on the sensor surface by the user.

22. The wearable sensor device of claim 21, wherein:
the first sensor includes at least one of a hydration sensor, a thermometer, an oximeter, a heart rate monitor, biosensor, a pedometer, a calorimeter, a watch, a biosensor, an accelerometer, a strain gauge, a blood glucose sensor, an oxygen sensor, an optical sensor, a heart rate monitor, moisture sensor, a positional sensor, and a rotational sensor; and
the second sensor includes at least one of a gyroscope, a compass, an accelerometer, an optical sensor, a proximity sensor, a thermometer, a pressure sensor, a force sensor, a camera, a microphone, and a microphone.

23. The wearable sensor device of claim 21, further comprising a device output module coupled to the analysis module and to the first sensor, and configured to generate output data that is based on the biological condition sensed by the first sensor while in operation in the first sensor regime or while in the second sensor regime.

24. The wearable sensor device of claim 21, wherein the first sensor includes one or more rings and a lead positioned on the sensor surface, wherein the one or more rings and the lead are configured to measure hydration levels using the sensor surface.

25. The wearable sensor device of claim 24, further comprising a circuit board, wherein the one or more rings and the lead are embedded in the circuit board.

26. The wearable sensor device of claim 25, further comprising:
a housing that encases the circuit board; and
a flexible strap that is attached to the housing.
